(19) United States
(12) Patent Application Publication
(10) Pub. No.: US 2019/0269876 A1
(43) Pub. Date: Sep. 5, 2019

(54) METHODS OF IMPROVING CRANIOFACIAL PAIN AND STROKE RECOVERY

(71) Applicant: AUTONOMIC TECHNOLOGIES, INC., Mountain View, CA (US)

(72) Inventors: William Hsu, Santa Clara, CA (US); Ian Welsford, Concord, CA (US); Ashlea Mittelstaedt, San Francisco, CA (US); Bruce Levin, Philadelphia, CA (US)

(21) Appl. No.: 16/288,485

(22) Filed: Feb. 28, 2019

Related U.S. Application Data
(60) Provisional application No. 62/636,895, filed on Mar. 1, 2018.

(51) Int. Cl. A61M 19/00; A61N 1/36

(52) U.S. Cl. CPC A61M 19/00; A61M 2205/054; A61M 2210/0618; A61N 1/36021

(57) ABSTRACT

Methods of improving craniofacial pain or stroke recovery via neurostimulation are provided. Methods include first screening the subject for neurostimulation based on the subject's responsiveness to a block of the sphenopalatine ganglion or branch thereof.

### CROSS-REFERENCE TO RELATED APPLICATIONS

The present application claims priority to U.S. Provisional Application No. 62/636,895, filed on Mar. 1, 2018, which is incorporated by reference in its entirety.

### TECHNICAL FIELD

Methods of improving medical conditions via neurostimulation by first screening the subject to determine if the subject is a suitable candidate for neurostimulation based on the subject's response to a SPG block are provided.

### BACKGROUND

The sphenopalatine ganglion (SPG) is a group of nerve cells located behind the bony structures of the nose. The nerve bundle is linked to the trigeminal nerve, the primary nerve involved in headache disorders. The SPG has both autonomic nerves, which are associated with functions such as tearing and nasal congestion, and sensory nerves, associated with pain perception.

Local anesthetics have been applied to the SPG region to block or partially block the SPG (“SPG blocks”) to reduce certain types of craniofacial pain such as cluster headaches. SPG blocks involve topical application of local anesthetic to mucosa overlying the SPG. The rationale for using SPG blocks to treat headaches is that local anesthetics in low concentrations could block the sensory fibers and thereby reduce pain while maintaining autonomic function. Originally, SPG blocks were done by inserting a cotton-tipped applicator dabbed with local anesthetic into the nose. Another variation is to insert a needle into the cheek and inject a local anesthetic. Other techniques involve inserting thin catheters into the nose to deliver numbing medication in and around the SPG.

In addition to the ganglion blockade using anesthetics, ablation (percutaneous radiofrequency, gamma knife, and surgical gangionectomy) have been used to treat pain (especially cluster headaches) originating in, or emanating from, the SPG. The objective of the ablation is to irreversibly damage the SPG to such an extent that it cannot generate the nerve signals that cause pain.

Another SPG intervention for certain types of pain, such as cluster headaches, is neurostimulation using an electrical neurostimulator to electrically stimulate the SPG. For example, an implantable microstimulator powered and controlled by radiofrequency waves generated by an external remote controller has been developed, whose electrode lead is positioned in the pterygopalatine fossa (PPF). Given that SPG stimulation is still a novel approach, there is currently no general consensus on patient selection and standards of care for SPG neuro stimulation.

### SUMMARY

The present disclosure relates to improving medical conditions in a subject via stimulation of a dorsonasal structure by screening the patient for neurostimulation using the results of an SPG block on the subject. In an aspect, a method of improving craniofacial pain in a subject suffering therefrom is provided. The method comprises applying an anesthetic to a first dorsonasal structure of the subject and determining if application of the anesthetic results in an improvement in craniofacial pain. The method further comprises identifying the subject as a candidate for neurostimulation upon a determination that the application of the anesthetic results in an improvement in the craniofacial pain. If the subject is a candidate for neurostimulation, the method comprises placing a neurostimulator adjacent to a second dorsonasal structure and activating the neurostimulator to deliver a therapy signal to the second dorsonasal structure to improve the subject's craniofacial pain. The first and second dorsonasal structures can be the same or different dorsonasal structures. In certain embodiments, the identifying step comprise identifying if the subject is a candidate for implantation of a neurostimulator and the placing step comprises implanting the neurostimulator adjacent to the second dorsal nasal structure.

In another aspect, a method of improving recovery from a stroke in a subject who has suffered from a stroke is provided. The method comprises applying an anesthetic to a first dorsonasal structure of the subject and determining if application of the anesthetic results in an improvement in stroke recovery. The method further comprises identifying the subject as a candidate for neurostimulation upon a determination that the application of the anesthetic results in an improvement in the stroke recovery. If the subject is a candidate for neurostimulation, the method comprises placing a neurostimulator adjacent to a second dorsonasal structure and activating the neurostimulator to deliver a therapy signal to the second dorsonasal structure to improve the subject's stroke recovery.

### BRIEF DESCRIPTION OF DRAWINGS

FIG. 1 is a flow diagram outlining steps of an exemplary method of the present disclosure.

### DETAILED DESCRIPTION

As used herein with respect to a described element, the terms “a,” “an,” and “the” include at least one or more of the described element including combinations thereof unless otherwise indicated. Further, the terms “or” and “and” refer to “and/or” and combinations thereof unless otherwise indicated. The terms “first,” “second,” etc. are used herein to describe various elements and are only used to distinguish one element from another. A “subject” can be a mammal and preferably is a human being. The present disclosure relates to improving certain medical conditions, such as craniofacial pain or stroke recovery, via neurostimulation, by first screening the subject based on the subject's response to a SPG block. Neurostimulation (also known as “neuromodulation”) is the alteration of nerve activity through targeted delivery of a stimulus, such as electrical stimulation or chemical agents, to specific neurological sites in the body. Such neuromodulation includes inhibiting or exciting the neural activity of the neurological site.

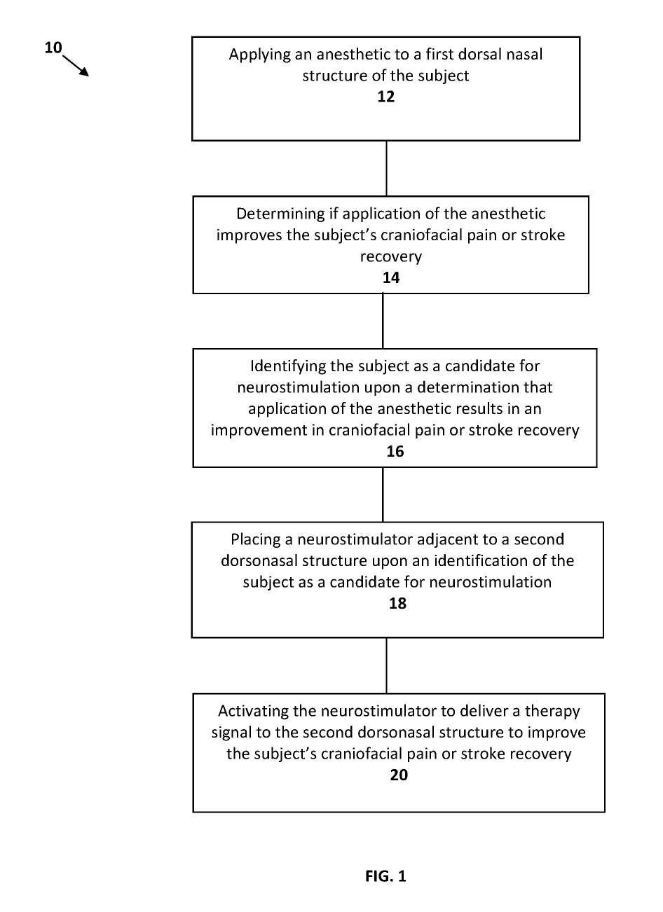

Referring to FIG. 1, in an embodiment, a method 10 for improving craniofacial pain or stroke recovery comprises applying an anesthetic to a first dorsal nasal structure of the subject 12. Method 10 further includes determining if application of the anesthetic improves the subject's craniofacial pain or stroke recovery 14. Method 10 further comprises identifying the subject as a candidate for neurostimulation upon a determination that the application of the anesthetic results in an improvement in craniofacial pain 16 or stroke recovery. If the subject is a candidate for neurostimulation, method 10 comprises placing a neurostimulator adjacent to a second dorsonasal structure 18 and activating the neurostimulator to deliver a therapy signal to the second dorsonasal structure to improve the subject's craniofacial pain or stroke recovery 20. The first and second dorsonasal structures can be the same or different dorsonasal structures. A dorsal nasal nerve structure includes a SPG, a sphenopalatine nerve (SPN) (also called the “pterygopalatine nerve”), a vidian nerve (VN) (also called “the nerve of the pterygoid canal”), a greater petrosal nerve (GPN), a lesser petrosal nerve, a deep petrosal nerve (DPN), or a branch thereof (e.g., a nasopalatine nerve, a greater palatine nerve, an inferior posterior lateral nasal branch of the greater palatine nerve, a lesser palatine nerve, or a superior maxillary nerve). The neurostimulator can be placed temporarily or permanently adjacent to the dorsonasal structure. In certain embodiments, a subject's response to an anesthetic is used to determine whether a subject is a candidate for an implantable neurostimulator. In such embodiments, if the patient is deemed to be a candidate for an implantable neuro stimulator, the step of placing a neurostimulator adjacent to the second dorsal nasal structure comprises implanting the neurostimulator adjacent to the second dorsal nasal structure.

The anesthetic that is applied to the first dorsonasal structure can be any suitable anesthetic that is used for blocking SPG activity to therapeutically alleviate craniofacial pain. Non-limiting examples of SPG blocks are lidocaine, bupivacaine, ropivacaine, levo-bupivacaine, ropivacaine, levo-ropivacaine, tetracaine, etidocaine, levo-etidocaine, dextro-etidocaine, levo-mepivacaine, pharmaceutically acceptable derivative thereof; a mixture of anesthetic agents and corticosteroids; and suitable combinations thereof. The SPG block can be administered by various approaches such as, for example, transnasal, transoral, and lateral infratemporal approaches.

Determining whether a subject is a candidate for neurostimulation can include determining if the anesthetic results in an improvement of craniofacial pain or stroke recovery. The improvement in the subject's pain can be determined by a variety of subjective and objective factors from either or both the physician and the subject, including ratings on pain scales. For example, an increase of five or more rating values on a pain scale from 0 to 10 can constitute an improvement in pain. Such scales are maintained by the American Headache Association, the American Chronic Pain Association and the International Pudendal Neuropathy Association. The comparative pain scale published by the last health association is often used in the treatment of migraines. Patients rate the pain associated with their migraine episodes on a scale from 0 to 10. TABLE I below provides a general description of each value on the scale.

TABLE I

| Rating | |
|---|---|
| Value | Description |
| 0 | Complete absence of pain |
| 1 | Slight discomfort; similar to a mosquito bite |
| 2 | Noticeable minor pain than can be easily forgotten |
| 3 | Sharp but manageable pain that does not last for too long |
| 4 | The difference between levels three and four is the level of |
| | distress. Toothaches are often rated at this level. |
| 5 | An example of pain at this level would be an ankle sprain that |
| | hurts each time a step is taken. |
| 6 | Some of the worst tension headaches are graded at this level, |
| | which involves the feeling of a painful force piercing the body. |
| 8 | Extremely intense pain |
| 9 | Throat cancer is one of the most unbearable disease conditions |
| | that is often rated at this pain level. |
| 10 | At this level, the pain is so overwhelming that a patient can |
| | lose consciousness or go in and out of shock. |

Migraine sufferers often rate their episodes at seven on the comparative scale above. The intensity of their pain can be exacerbated by strong feelings of nausea and be rated at eight or nine. Another self-reporting instrument is the neuropathic pain scale, which also ranges from 0 to 10 and adds pain descriptors such as levels of sharpness, itchiness and warmth. This scale also differentiates between constant, background, flaring, and occasional pain. On a 5 point scale, where 4 is very severe pain; 3 is severe pain; 2 is moderate pain; 1 is mild pain; and 0 is no pain, an improvement in craniofacial pain can result in a level 4 or 3 reduction to 1 or 0 or a level 2 reduction to 0 measured approximately 15 minutes post-intervention.

With respect to stroke recovery, improvements can include improvements in motor, cognitive, or speech function for example. An improvement in a patient's recovery can include one or more measurable (e.g., objective or subjective) improvements of at least one variable in a patient having suffered a stroke as compared to a baseline or control value. Variables in which an improvement can be measured include, but are not limited to, basic self needs (e.g., bathing, cooking, eating, dressing, grooming, writing, using a computer, holding a conversation) and more complex tasks, such as complex reasoning, memory, judgment and driving. Other methods for measuring improvements in a patient's recovery from a stroke are known in the art. In some instances, the baseline or control value can be obtained from an apparently healthy subject such as a subject who has not suffered from a stroke or a population of apparently healthy subjects. In other instances, the baseline or control value can be obtained from a patient or population of patients before the patient or population of patients experiences a stroke. In still other instances, the baseline or control value can include measurements, taken at various times, from a patient or population of patients that has/have suffered from a stroke.

Without wishing to be bound by theory, it is believed that if a subject experiences effective acute relief or recovery from an SPG block, it is likely that a dorsonasal structure, such as the SPG, is involved in the subject's craniofacial pain or stroke recovery. However, because the relief or recovery may only be short-term, it is not practical for the subject to continuously repeat the SPG block procedure. Therefore, a neurostimulator, such as an implantable neurostimulator, which delivers a therapy signal to the dorsonasal structure can be a viable choice. Such a methodology for determining whether a subject is a suitable candidate for a neurostimulator is currently not used in clinical settings. Physicians are debating if the mechanisms underlying craniofacial pain, such as headaches, that are affected by an SPG block are the same as those mechanisms that will be affected by neurostimulation, such as electrical stimulation of the SPG. Such a methodology can improve the responder rate of the neurostimulation by screening for patients who can benefit from this therapy.

In addition to determining whether a subject is a candidate for neurostimulation, the results of the SPG block also can be used to determine the location and/or programming setting of the neurostimulator to maximize therapy. For instance, if the relief is only short term, the neurostimulator can be placed as close to the dorsonasal structure as possible to maximize the stimulation of the dorsonasal structure. Further, in such circumstances, the programming settings will likely to be set high.

In certain embodiments, an improvement can be short-term such as an improvement in pain for a period of less than approximately 30 minutes but greater than 0 seconds. In other embodiments, no improvement or a minor improvement in craniofacial pain or stroke recovery can be used to determine if a subject is a candidate for neurostimulation. As stated above, improvement in a subject's pain can be determined by a variety of subjective and objective factors from either or both the physician and subject, including ratings on pain scales. For example, an increase of less than 3 rating values on a pain scale from 0 to 10 can constitute no improvement or a minor improvement in craniofacial pain. Such a result can be useful when the physician believes that the SPG block was not performed correctly (e.g. the technique, volume concentration or timing of the block was inadequate) or because there is a subset of patients where the pharmakinetic effect of the anesthetic is different from the effect that is obtained from neurostimulation. In other embodiments, a long-term improvement in craniofacial pain or stroke recovery can be used to determine if a subject is a candidate for neurostimulation. A long-term improvement is an improvement in pain for a period of greater than approximately 1 day.

If the subject is deemed a candidate for neurostimulation, a method comprises placing a neurostimulator adjacent to a second dorsonasal structure. The neurostimulator is placed adjacent to the second dorsonasal structure such that the neurostimulator is sufficiently close to the second dorsonasal structure to directly modulate the second dorsonasal structure (as opposed to modulating a proximal, upstream nerve that innervates the dorsonasal structure or a distal, downstream nerve that is innervated by the dorsonasal structure). In certain embodiments, the neurostimulator is placed in the craniofacial region of the subject. In certain embodiments, the neurostimulator is placed in the PPF. In certain embodiments, the neurostimulator is placed in direct contact with an SPG. The neurostimulator can be placed transcutaneously, trans-orally, trans-nasally, percutaneously, subcutaneously, intraluminally, or intravascularly adjacent to the dorsonasal structure. In particular, there are several surgical approaches to the PPF that may be used to deliver a neurostimulator into the PPF such as a gingival-buccal approach; a trans-oral approach, with a dental needle up to the sphenopalatine foramen through the posterior palatine canal; a lateral approach to the PPF through the infratemporal fossa; an infrazygomatic approach, in which the skin entry is at a site overlying the PPF, inferior to the zygoma and anterior to the mandible. Other approaches include a trans-nasal approach through the nasal cavity; and other routes through the mouth and outer skin of the face.

Once the neurostimulator is placed adjacent to the second dorsonasal structure, a method comprises activating the neurostimulator to deliver a therapy signal to the second dorsonasal structure. A neurostimulator can be configured or programmed to deliver various types of therapy signals to the second dorsonasal structure. For example, a neurostimulator can be configured or programmed to deliver only electrical energy (e.g. an electrical therapy signal), only a pharmacological or biological agent (e.g. a chemical therapy signal), or a combination thereof. In one example, a neurostimulator can comprise at least one electrode and an integral or remote electrical energy generator which is in electrical communication with the one or more electrodes and configured to produce one or more electrical signals (or pulses). In another example, a neurostimulator can include a pharmacological or biological agent reservoir, a pump, a fluid dispensing mechanism, or a long-lasting polymer that encapsulates the drug in the form of a pellet, sheet or other form, for example, to allow slow infusion or delivery of medications and other agents that can modulate the dorsonasal structure. Non-limiting examples of pharmacological and biological agents include chemical compounds, drugs, nucleic acids, polypeptides, stem cells.

The neurostimulator can also be configured or programmed to deliver various other energy forms within the energy spectrum and/or biological forms of therapy, such as, for example, sound waves, ultrasound, radiofrequency (continuous or pulsed), optical, infrared, microwave, magnetic waves, cryotherapy, heat, or optogenetic therapy. The neurostimulator can also apply mechanical forms of therapy, such as pressure.

An electrode of a neurostimulator can be controllable to provide output signals that may be varied in voltage, amplitude, frequency, pulse-width, current and intensity. The electrode can also provide both positive and negative current flow from the electrode and/or is capable of stopping current flow from the electrode and/or changing the direction of current flow from the electrode. In some instances, a neurostimulator can include an electrode that is controllable, i.e., in regards to producing positive and negative current flow from the electrode, stopping current flow from the electrode, changing direction of current flow from the electrode, and the like. In other instances, the electrode has the capacity for variable output, linear output and short pulse-width. In other instances, the electrode can comprise a coil configured to deliver magnetic stimulation.

A therapy signal that is an electrical signal may be constant, intermittent, varying and/or modulated with respect to the current, voltage, pulse-width, waveform, cycle, frequency, amplitude, and so forth. The stimulation may be continuous or of intermittent durations. The electrode may be mono-polar, bipolar or multi-polar of any suitable configuration and geometry to accommodate stimulation of the dorsonasal structure.

A controller or programmer may also be associated with a neurostimulator. A programmer, for example, can include one or more microprocessors under the control of a suitable software program. The programmer can include other components such as an analog-to-digital converter, etc.

Neurostimulators can be pre-programmed with desired stimulation parameters. Stimulation parameters can be controllable so that a therapy signal may be remotely modulated to desired settings without removal of the neurostimulator from its target position. Remote control may be performed, e.g., using conventional telemetry with an implanted electric signal generator, an implanted radiofrequency receiver coupled to an external transmitter, and the like. In some instances, some or all parameters of the neurostimulator may be adjusted manually by the physician or under the control or supervision of a physician. In other instances, some or all parameters of the neurostimulator may be automatically controllable by a programmer or controller comprising the neurostimulator. In certain embodiments, the neurostimulator is programmed to deliver electrical energy to the dorsonasal structure in biphasic charge-balanced pulses having a frequency of about 1-1000 Hz (e.g., 5-200 Hz), a pulse-width of about 0.04-2 ms, a current of about 0.05-100 mA (e.g., 0.1-5 mA), and a voltage of about 1-10 V.

Neurostimulators can be part of an open- or closed-loop system. In an open-loop system, for example, a physician or subject may, at any time, manually or by the use of pumps, motorized elements, etc., adjust treatment parameters, such as pulse amplitude, pulse-width, pulse frequency, duty cycle, dosage amount, type of pharmacological or biological agent, etc. Alternatively, in a closed-loop system, treatment parameters may be automatically adjusted in response to a sensed physiological parameter or a related symptom indicative of the extent of the craniofacial pain or stroke recovery. In a closed-loop feedback system, a sensor that senses a physiological parameter associated with the craniofacial pain or motor/cognitive/speech function of the subject in the case of stroke can be utilized. Sensors to measure physiological parameters can be external of the patient's body, or on the patient's body.

In certain embodiments, a neurostimulator can be part of a system that also includes include a remote transducer, a personal electronic device and, optionally, a programming device. Each component of the system can be in communication (e.g., electrical communication) with one another. In some instances, two or more components of the system can be in wireless communication with one another. In other instances, two or more components of the system can be in wired communication with one another. It will be appreciated that some components of the system can be in wireless communication with one another while other components are in wired communication with one another.

The neurostimulator can comprise electronic circuitry and one or more electrodes that is/are driven by the circuitry, and one or more transmit coils, radiators, or PCB antennas (not shown). The electronic circuitry of the neurostimulator can be programmed to receive and transmit data (e.g., stimulation parameters) and/or power from outside the body. In some instances, the electronic circuitry can include a programmable memory for storing at least one set of stimulation and control parameters. In other instances, the neurostimulator can include a power source and/or power storage device. Possible power options can include, but are not limited to, various wireless charging mechanisms, such as an external power source coupled to the neurostimulator via an RF link using coils or radiators, a self-contained power source utilizing any means of generation or storage of energy (e.g., a primary battery, a rechargeable battery, such as a lithium ion battery, a button or coin cell battery, an electrolytic capacitor, or a super- or ultra-capacitor), and, if the self-contained power source is rechargeable, a mechanism for recharging the power source (e.g., an RF link). In some instances, the system can include a retractable power cable that can be selectively connected to the power source and/or power storage device.

In the case where methods are used to improve craniofacial pain with a neurostimulator that is a drug delivery device, the drug can be methysergide; propanolol; a calcium channel blocker such as verapamil; an ergotamine preparation such as dihydroergotamine; a serotonin receptor agonist such as sumatriptan, zolmitriptan, and rizatriptan; aspirin; codeine; a vasocontrictor; a narcotic; butorphanol tartrate; meperidine; a corticosteroid; oxygen; indomethacin; topiramate; lithium; or a suitable combination of these compounds. In the case where methods are used to improve stroke recovery with a neurostimulator that is a drug delivery device, the drug can be serotonergic drugs including antidepressants, dopaminergic drugs, noradrenergic drugs, cholinergic drugs, amphetamines, or suitable combinations thereof.

Non-limiting examples of craniofacial pain include pain from migraine headaches including acute and chronic migraine headaches with aura, migraine headaches without aura, menstrual migraines, migraine variants, atypical migraines, complicated migraines, hemiplegic migraines, transformed migraines, and chronic daily migraines; episodic tension headaches; chronic tension headaches; analgesic rebound headaches; episodic cluster headaches; chronic cluster headaches; cluster variants; chronic paroxysmal hemicrania; hemicrania continua; post-traumatic headache; post-traumatic neck pain; post-herpetic neuralgia involving the head or face; pain from spine fracture secondary to osteoporosis; arthritis pain in the spine, headache related to cerebrovascular disease and stroke; headache due to vascular disorder; reflex sympathetic dystrophy, cervicalgia (which may be due to various causes, including, but not limited to, muscular, discogenic, or degenerative, including arthritic, posturally related, or metastatic); glossodynia, carotidynia; cricoidynia; otalgia due to middle ear lesion; gastric pain; sciatica; maxillary neuralgia; laryngeal pain, myalgia of neck muscles; trigeminal neuralgia (sometimes also termed tic douloureux); post-lumbar puncture headache; low cerebro-spinal fluid pressure headache; temporomandibular joint disorder; atypical facial pain; ciliary neuralgia; paratrigeminal neuralgia (sometimes also termed Raeder's syndrome); petrosal neuralgia; Eagle's syndrome; idiopathic intracranial hypertension; orofacial pain; myofascial pain syndrome involving the head, neck, and shoulder; chronic migraneous neuralgia, cervical headache; paratrigeminal paralysis; sphenopalatine ganglion neuralgia (sometimes also termed lower-half headache, lower facial neuralgia syndrome, Sluder's neuralgia, and Sluder's syndrome); carotidynia; Vidian neuralgia; and causalgia; or a combination of the above.

Non-liming examples of stroke include ischemic stroke, a hemorrhagic stroke such as a subarachnoid hemorrhage or an intracerebral hemorrhage; or a transient ischemic attack. In the instance of a subarachnoid hemorrhage, methods can be used to improve cerebral vasospasms or delayed cerebral ischemia. In such instances, the dorsonasal structure can be the SPG, for example.

Although the present disclosure is described with respect to craniofacial pain and stroke, the methods can be used for other medical conditions that can be mediated by dorsonasal structures such as hypertension and disorders included in U.S. Pat. No. 6,526,318, which is incorporated by reference in its entirety. In embodiments of methods of improving hypertension, a responder to a SPG block can be determined by monitoring the subject's blood ambulatory blood pressure a week prior to the SPG block procedure as well as during a period of between approximately 21 to approximately 30 days after the SPG block in order to estimate differences in 24 hour average systolic (24 hour SBP) and diastolic blood pressure (24 hour DBP), daytime, nighttime, pre-awake and early morning SBP and DBP as well as BP load. A candidate for neurostimulation can be a subject that is a responder to an SPG block. As responder to an SPG block can be a subject where there is a 24 hour SBP decrease of >5 mmHg, a SBP and DBP reduction during the overall 24 hour period, a reduction in SBP and DBP during daytime and nighttime periods, a reduction in pre-awake SBP and/or daytime SBP and DBP load decrease.

Each of the disclosed aspects and embodiments of the present disclosure may be considered individually or in combination with other aspects, embodiments, and variations of the disclosure. Unless otherwise specified, none of the steps of the methods of the present disclosure are confined to any particular order of performance.

What is claimed is:

1. A method of improving craniofacial pain in a subject suffering therefrom comprising:
applying an anesthetic to a first dorsonasal structure of the subject;
determining if application of the anesthetic results in an improvement in craniofacial pain;
identifying the subject as a candidate for neurostimulation upon a determination that application of the anesthetic results in an improvement in the craniofacial pain;
placing a neurostimulator adjacent to a second dorsonasal structure upon an identification of the subject as a candidate for neurostimulation;
activating the neurostimulator to deliver a therapy signal to the second dorsonasal structure; and
improving the subject's craniofacial pain via activation of the neurostimulator.

2. The method of claim 1, wherein the first dorsonasal structure and the second dorsonasal structure are the same structure.

3. The method of claim 1, wherein the first dorsonasal structure and the second dorsonasal structure are different structures.

4. The method of claim 1, wherein the neurostimulator is an electrical neurostimulator and the therapy signal is an electrical signal.

5. The method of claim 1, wherein the neurostimulator is a drug delivery device and the therapy signal is a chemical signal.

6. The method of claim 1, wherein the dorsonasal structure is a sphenopalatine ganglion.

7. The method of claim 1, wherein the identifying step comprise identifying if the subject is a candidate for implantation of a neurostimulator and wherein the placing step comprises implanting the neurostimulator adjacent to the second dorsal nasal structure.

8. The method of claim 1, wherein the determining step comprises determining if application of the anesthetic results in a short-term improvement in the craniofacial pain.

9. A method of improving stroke recovery in a subject who has suffered from stroke comprising:
applying an anesthetic to a first dorsonasal structure of the subject;
determining if application of the anesthetic results in an improvement in the stroke recovery;
identifying the subject as a candidate for neurostimulation upon a determination that the application of the anesthetic results in an improvement in the stroke recovery;
placing a neurostimulator adjacent to a second dorsonasal structure upon an identification of the subject as a candidate for neurostimulation;
activating the neurostimulator to deliver a therapy signal to the second dorsonasal structure; and
improving the subject's stroke recovery via activation of the neurostimulator.

10. The method of claim 9, wherein the first dorsonasal structure and the second dorsonasal structure are the same structure.

11. The method of claim 9, wherein the first dorsonasal structure and the second dorsonasal structure are different structures.

12. The method of claim 9, wherein the neurostimulator is an electrical neurostimulator and the therapy signal is an electrical signal.

13. The method of claim 9, wherein the neurostimulator is a drug delivery device and the therapy signal is a chemical signal.

14. The method of claim 9, wherein the dorsonasal structure is a sphenopalatine ganglion.

15. The method of claim 9, wherein the identifying step comprise identifying if the subject is a candidate for implantation of a neurostimulator and wherein the placing step comprises implanting the neurostimulator adjacent to the second dorsal nasal structure.
